(19) United States
(12) Patent Application Publication
(10) Pub. No.: US 2004/0180376 A1
(43) Pub. Date: Sep. 16, 2004

(54) Efficient test of association for quantitative traits and affected-unaffected studies using pooled DNA

(76) Inventors: Joel S. Bader, Stamford, CT (US); Aruna Bansal, Cambridgeshire (GB); Pak Sham, London (GB)

Correspondence Address: Ivor R. Elrifi, Mintz, Levin, Cohn, Ferris, Glovsky and Popeo, P.C, One Financial Center, Boston, MA, 02111, US

(21) Appl. No.: 10/815,062

(22) Filed: Mar. 30, 2004

Related U.S. Application Data
(63) Continuation of application No. 09/973,449, filed on Oct. 9, 2001, now abandoned.
(60) Provisional application No. 60/238,381, filed on Oct. 6, 2000.

(51) Int. Cl. C12Q 1/68; A01H 1/00

(52) U.S. Cl. 435/6; 800/278

(57) ABSTRACT

Risk assessment and diagnosis of a complex disorder often requires measuring an underlying quantitative phenotype. Association studies in unrelated populations can implicate genetic factors contributing to disease risk, and experiments using pooled DNA provide a less costly but necessarily less powerful alternative to methods based on individual genotyping. Although the sample sizes required for pooling and individual genotyping studies have been compared in certain instances, general results have not been reported in the context of association studies, nor have there been clear comparisons of pooling based on quantitative and qualitative (affected/unaffected) phenotypes. Here we use exact numerical calculations and analytical approximations to examine the sample size requirements of association tests for quantitative traits and affected-unaffected studies using pooled DNA. We show, in analogy with selection experiments, that the optimal design for virtually any quantitative phenotype is to pool the top and bottom 27% of individuals, regardless of marker frequency or inheritance mode; this design requires a population only 24% larger than that required for individual genotyping. Furthermore, this design is approximately four times more efficient than typical affected-unaffected studies of DNA pooled from individuals classified as affected or unaffected.

### RELATED APPLICATION

This application claims priority to U.S. Ser. No. 60/238,381, filed Oct. 6, 2000 [21402-139] which is incorporated herein by reference in its entirety.

### BACKGROUND OF THE INVENTION

The complex diseases that present the greatest challenge to modem medicine, including cancer, cardiovascular disease, and metabolic disorders, arise through the interplay of numerous genetic and environmental factors. One of the primary goals of the human genome project is to assist in the risk-assessment, prevention, detection, and treatment of these complex disorders by identifying the genetic components. Disentangling the genetic and environmental factors requires carefully designed studies. One approach is to study highly homogenous populations (Nillson and Rose 1999; Rabinow, 1999; Frank 2000). A recognized drawback of this approach, however, is that disease-associated markers or causative alleles found in an isolated population might not be relevant for a larger population. An attractive alternative is to use well-matched affected-unaffected studies of a more diverse population Even with a well-matched sample set, the genetic factors contributing to an aberrant phenotype may be difficult to determine. Traditional linkage analysis methods identify physical regions of DNA whose inheritance pattern correlates with the inheritance of a particular trait (Liu 1997; Sham 1997, Ott 1999). These regions may contain millions of nucleotides and tens to hundreds of genes, and identifying the causative mutation or a tightly linked marker is still a challenge. A more recent approach is to use a sufficiently dense. marker set to identify causative changes directly. Single nucleotide polymorphisms, or SNPs, can provide such a marker set (Cargill et al. 1999). These are typically bi-allelic markers with linkage disequilibrium extending an estimated 10,000 to 100,000 nucleotides in heterogeneous can provide such a marker set (Cargill et al. 1999). These are typically bi-allelic markers with linkage disequilibrium extending an estimated 10,000 to 100,000 nucleotides in heterogeneous human populations (Kruglyak 1999; Collins et al. 2000). Tens to hundreds of thousands of these closely spaced markers are required for a complete scan of the 3 billion nucleotides in the human genome. Because each SNP constitutes a separate test, the significance threshold must be adjusted for multiple hypotheses (p-value ˜10⁻⁸) to identify statistically meaningful associations. Consequently, hundreds to thousands of individuals are required for association studies (Risch and Merikangas 1996).

The most powerful tests of association require that each individual be genotyped for every marker (Fulker et al. 1995, Kruglyak and Lander 1995, Abecasis et al. 2000, Cardon 2000) and remain far too costly for all but testing candidate genes. An alternative that circumvents the need for individual genotypes, related to previous DNA pooling methods for determination of linkage between a molecular marker and a quantitative trait locus (Darvasi and Soller 1994), is to determine allele frequencies for sub-populations pooled on the basis of a qualitative phenotype. Populations of unrelated individuals, separated into affected and unaffected pools, have greater power than related populations. Limited guidance has been provided, however, regarding the sample size requirement of tests using pooled DNA relative to individual genotyping, or the efficiency of tests based on a quantitative phenotype relative to an affected/unaffected design.

The phenotypes relevant for complex disease are often quantitative, however, and converting a quantitative score to a qualitative classification represents a loss of information that can reduce the power of an association study. The location of the dividing line for affected versus unaffected classification, for example, can affect the power to detect association. Furthermore, pooling designs based on a comparison of numerical scores are not even possible with a qualitative classification scheme. These distinctions can be especially relevant when populations contain related individuals and qualitative tests have a disadvantage (Risch and Teng 1998).

When performing risk assessment to determine whether a person suffers from or is at risk of developing a complex disorder often requires measuring an underlying quantitative phenotype. Association studies in unrelated populations can implicate genetic factors contributing to disease risk, and experiments using pooled DNA provide a less costly but necessarily less powerful alternative to methods based on individual genotyping. Association studies require markers in linkage disequilibrium with causative genetic polymorphisms. Although the sample sizes required for pooling and individual genotyping studies have been compared in certain instances, general results have not been reported in the context of association studies, nor have there been clear comparisons of pooling based on quantitative and qualitative (affected/unaffected) phenotypes. Association tests of DNA pooled on the basis of a quantitative phenotype are analogous to selection experiments for quantitative trait locus (QTL) mapping. For a QTL with a weak effect on a phenotype, the mean phenotypic value of individuals selected to exceed a threshold is proportional to the mean allele enrichment. This suggests that genotyping of a certain percentage of the upper and lower phenotypic values of an unrelated population is useful to estimate the effect of a marker on a quantitative phenotype, such as in pooling studies. There is a need in the art to examine the sample size requirements of association tests for quantitative traits using pooled DNA.

### SUMMARY OF THE INVENTION

The present invention is based, in part, on the discovery of methods to detect an association in a population of individuals between a genetic locus and a quantitative phenotype, where two or more alleles occur at a given genetic locus, and the phenotype is expressed using a numerical phenotypic value whose range falls within a first numerical limit and a second numerical limit. These limits are used to provide for subpopulations that consist of upper and lower pools.

In some embodiments, the population of individuals includes individuals who may be classified into classes. In certain aspects of the invention, these classes are based on age, gender, race, or ethnic origin. In other aspects, some or all members of a class are included in the pools.

In various embodiments, these numerical limits are chosen so that the upper pool includes the highest 19%, 27%, or 37% of the population. In other embodiments, the numerical limits are chosen such that the lower pool includes the lowest 19%, 27%, or 37% of the population.

In some embodiments, the upper and lower pools have the same number of individuals.

In one embodiment of the invention, the numerical limits are chosen to correlate with error of measurement determinations. In some embodiments, the numerical limit on the error of measurement is about 0.04 or about 0.01.

In some embodiments, methods to detect an association in a population of individuals between a genetic locus and a quantitative phenotype are useful to determine the genetic basis of disease predisposition.

In other embodiments, the genetic locus analyzed contains a single nucleotide polymorphism.

In the present invention, the population of individuals can include unrelated individuals.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE FIGURES

FIG. 1. The sample size required to achieve a type I error rate of 5×10⁻⁸ and a power of 0.8 for a QTL for a complex trait is shown for pooled DNA designs relative to individual genotyping. The ratio N_{c-c}/N_{indiv} for affected-unaffected pools (dashed line) is shown as a function the disease incidence r, while the ratio Nₜₐᵢₗ/N_{indiv} (solid line) is shown as a function of the fraction ρ of the total population selected for each pool. The optimum value of Nₜₐᵢₗ/N_{indiv} is 1.24, occurring at ρ=27% selected for each pool.

FIG. 2a Exact numerical results for the sample size N required to achieve a type I error rate of 5×10⁻⁸ with a power of 0.8 are shown for affected-unaffected pools (dashed line) and tail pools (solid line) as a function of the additive variance, or equivalently the genotype relative risk for a heterozygote, for an allele with frequency 0.1 and purely additive variance. Analytic approximations (solid circles), Eqs. 1 and 2, are indistinguishable from the exact results when the genotype relative risk is smaller than a factor of 2. The disease incidence r is 10% for the affected-unaffected pools, and 27% of the population is selected for the each of the tail pools.

FIG. 2b The frequency difference at the significance threshold is shown for the same parameters as panel a. This threshold determines the measurement accuracy required for an association test based on pooled DNA.

### DETAILED DESCRIPTION OF THE INVENTION

The present invention provides analytic results for association tests. It is shown that the results obtained closely approximate the analytic results to exact numerical calculations. The invention further extends the analysis to qualitative phenotypes using a genotype relative risk model.

A particular quantitative phenotype X is standardized to have unit variance and zero mean. The phenotype is hypothesized to be affected by alleles A₁ and A₂, with frequencies p and 1−p respectively, at a particular QTL. The population fractions P(G) for genotypes G=A₁A₁, A₁A₂, and A₂A₂ are assumed obey Hardy-Weinberg equilibrium. Using standard notation for a variance components model (Falconer and MacKay, 1996), the effect μ_{G} of genotype G on phenotype X is a−μ for A₁A₁, d−μ for A₁A₂, and −a−μ for A₂A₂. The constant μ=(2p−1)a+2p(1−p)d ensures that the mean of X is zero. The ratio d/a describes the inheritance mode for allele A₁. Dominant, recessive, and additive inheritance are special cases with d/a equal to +1, −1, and 0, respectively.

The phenotypic variance due to the QTL may be partitioned into the additive variance σ_{A}² and the dominance variance σ_{D}², with

σ_{A}²+σ_{D}²=2pq[a−d(p−q)]²+4p²q²d².

The additive variance is often much larger than the dominance variance even if the inheritance mode is not purely additive. The exceptions are QTLs with a recessive minor alleles and dominant major alleles, which are difficult to detect in unselected populations. The contribution of remaining genetic and environmental factors is assumed to follow a normal distribution with residual variance σ_{R}²,

σ_{R}²=1−(σ_{A}²+σ_{D}²).

Of particular interest here are complex traits: the effect of any single QTL is small, σ_{A}²+σ_{D}²<0.05, and the residual variance σ_{R}² is nearly 1.

A genotype relative risk model corresponds to classifying individuals as affected (X>X_{T}) or unaffected (X<X_{T}) based on a specific threshold X_{T}. The proportion r of the total population that is affected is the overall risk or disease incidence; the probability that an individual with genotype G is affected, relative to the probability for an individual with genotype A₂A₂, is the genotype relative risk. If the inheritance mode of A₁ is additive and a is small compared to σ_{R}, the relative risk is multiplicative with allele dose.

The sample size N required to detect association between genotype G and the quantitative phenotype or the disease risk depends on the type I error rate α, the type II error rate β, and the test statistic and experimental design (Snedecor and Cochran, 1989), as well as on the underlying genetic model. For a one-sided test of a single marker, α=1−Φ(z_{α}), where Φ(z) is the cumulative probability distribution for standard normal deviate z, defines α in terms of deviate z_{α}. Similarly, 1−β is the power to reject the null hypothesis and z_{1-β}=Φ⁻¹ (β). For a genome scan, the values α=5×10⁻⁸ (z_{α}=5.33) and 1−β=0.8 (z_{1-β}=−0.84) have been suggested (Risch and Merikangas, 1996).

We consider two experimental designs using DNA pooled from individuals selected from a sample of size N: affected-unaffected pools, with DNA pooled from n affected and n unaffected individuals; and tail pools, with DNA pooled from n individuals at each tail of the phenotype distribution. The test statistic for these designs is the frequency difference of the A₁ allele between the pools. The multinomial distribution describing the test statistic may be used to calculate exactly the sample size required to achieve statistical significance at specified power.

When the number of A₁ alleles summed over both pools is large, the distribution of the test statistic is approximately normal. A significant association is detected if the allele frequency difference between pools is at least z_{α} times the standard deviation of its estimator, or z_{α}p^{1/2}(1−p)^{1/2}/n^{1/2}. Furthermore, when the additive variance σ_{A}² is small and the residual variance σ_{R}² is close to 1, convenient analytic approximations for the sample size requirements may be derived.

For the affected-unaffected design, n=rN of the individuals are expected to be diagnosed as affected, and an additional n matched controls are selected from the remainder of the population. The analytic approximation for the sample size is

N_{c-c}=[z_{α}−z_{1-β}]²[σ_{R}²/σ_{A}²]·2r(1−r)²/y²[1+X_{T}(1−σ_{R}²)^{1/2}/2^{3/2}σ_{R}²p^{1/2}(1−p)^{1/2}]². (Eq. 1)

The term y is the height of the standard normal distribution at the normal deviate X_{T}/σ_{R} corresponding to the threshold between affected and unaffected phenotypic values.

The tail pools are parameterized by the fraction ρ=n/N of population selected for each pool, and ρ plays a role analogous to the overall disease incidence r in the affected-unaffected design. The analytical approximation for the sample size is

Nₜₐᵢₗ=[z_{α}−z_{1-β}]²[σ_{R}²/σ_{A}²]·ρ/2y², (Eq. 2)

where y is the height of the standard normal distribution for normal deviate Φ⁻¹ (ρ). The design may be optimized by selecting ρ to minimize Nₜₐᵢₗ, which corresponds to minimizing ρ/2y². With this approximation, the optimal fraction is 0.27 and is independent of α, β, and all parameters of the genetic model.

A third method, individual genotyping, serves as a baseline for evaluating the efficiency of the two pooling-based methods. The sample size required to achieve significance using individual genotyping is

N_{indiv}=[z_{α}−z_{1-β}σ_{R}]²/σ_{A}², (Eq. 3)

based on a regression model of phenotypic value on allele dose.

Detailed Description of Analytical Methods

The genotype-dependent phenotype distribution in the variance components model is

P(X|G)=(2π)^{-1/2}exp[−(X−μ_{G})²/σ_{R}²],

and the overall phenotype distribution is the sum of the three normal distributions,

P(X)=Σ_{G}P(X|G)P(G).

When an upper threshold X_{U} is specified to select a fraction ρ of the total population with phenotypic values above the threshold, the equation

ρ=Σ_{G}{1−Φ[(X_{U}−μ_{G})/σ_{G}]}P(G).

may be solved numerically for X_{U} as a function of ρ. The genotypes of individuals selected by X>X_{U} follow a multinomial distribution; the probability that an individual has genotype G is θ_{U}(G)={1−Φ[(X_{U}−μ_{G})/σ_{G}]}P(G)/ρ. A multinomial distribution is similarly defined using a lower threshold X_{L},

1=Σ_{G}θ_{L}(G)=ρ⁻¹Σ_{G}Φ[(X_{L}−μ_{G})/σ_{G}]P(G).

For an affected-unaffected design, the fraction in the upper pool is r and the fraction in the lower pool is 1−r, yielding X_{U}=X_{L}=X_{T}. The relative risk for genotype G is [θ_{U}(G)/P(G)]/[θ_{U}(A₂A₂)/P(A₂A₂)].

Sample size requirements may be obtained directly from the multinomial distributions of genotypes by exhaustively tabulating allele counts C_{U} and C_{L} in the upper and lower pools for each distinct composition of genotypes among the n selected individuals. The distribution corresponding to null hypothesis, θ(G)=P(G), is used to define the smallest threshold ΔC such that C_{U}−C_{L}≦ΔC with probability a or less. The discrete allele count usually yields the strict inequality. Next, the distributions under the alternative hypothesis are considered, and the probability that C_{U}−C_{L}≦ΔC is tabulated to provide the power. If the power is greater than or equal to the specified 1−β, the choice of n and N=n/ρ or n/r is feasible. A search is performed for the smallest feasible N with r or ρ specified. For tail pools, ρ is then varied to find the overall optimum.

When the number of alleles summed over both pools is large, the allele frequency difference follows a normal distribution. Under the null hypothesis, the mean is zero and variance is σ₀²/n=p(1−p)/n. This result is derived by noting that the variance of the frequency difference is twice the variance of the mean for a single pool of n individuals. The allele frequency variance for an individual is p(1−p)/2, and averaging over the n individuals reduces the variance by the factor n. Under the alternative hypothesis, the expected allele frequency difference Δp is

Δp=p_{U}−p_{L}=Σ_{G}[θ_{U}(G)−θ_{L}(G)]p_{G}

where the genotype-dependent allele frequency p_{G} is 1 for G=A₁A₁, 0.5 for A₁A₂, and 0 for A₂A₂. The variance is σ₁²/n, where σ₁² is obtained from the multinomial distribution (Beyer, 1984),

σ₁²=Σ_{G}[θ_{U}(G)+θ_{L}(G)]p_{G}²−(p_{U}²+p_{L}²).

The number of individuals required per pool for type I error α and power 1−β is

n=[z_{ασ}₀−z_{1-β}σ₁]²/Δp².

For affected-unaffected pools, N=n/r is the required sample size. For tail pools, N=n/ρ, and ρ is varied to find the smallest N.

The normal approximation underestimates the sample size requirement relative to the exact results from the multinomial distribution. When the sum of the alleles in both pools is at least 60, the difference in sample sizes is no greater than 5%. We chose 60 alleles in both pools as the criterion for switching from the multinomial to the normal calculation. Standard algorithms were employed to perform the root search for X_{U} and X_{L}, the optimization, and the integration over the tail of a normal distribution (Press, 1997).

The analytic results are obtained by setting σ₁² to σ₀² and expanding Δp to second order in the effect size μ_{G}, corresponding loosely to a perturbation theory for probability distributions (Chandler, 1987). From a Taylor series expansion,

Φ(z−b)=Φ(z)−by−(1/2)b²yz,

where y=(2π)^{-1/2}exp(−z²/2). Substituting this result into the expressions for θ(G) using b=μ_{G}/σ_{R} and z=X_{U}/σ_{R}=Φ⁻¹(1−ρ), where X is the threshold used to select the pool, yields for the tail design

p_{U}=p+(y/ρ)E[(μ_{G}/σ_{R})p_{G}]+(y|z|/2ρ)E[(μ_{G}/σ_{R})²p_{G}] and

p_{L}=p−(y/ρ)E[(μ_{G}/σ_{R})p_{G}]+(y|z|/2ρ)E[(μ_{G}/σ_{R})²p_{G}].

The corresponding results for the affected-unaffected pools, with z=Φ⁻¹(1−r), are

p_{U}=p+(y/r)E[(μ_{G}/σ_{R})p_{G}]+(y|z|/2r)E[(μ_{G}/σ_{R})²p_{G}] and

p_{L}=p−[y/(1−r)]E[(μ_{G}/σ_{R})p_{G}]−[y|z|/2(1−r)]E[(μ_{G}/σ_{R})²p_{G}].

The required expectation values are

E[μ_{G}p_{G}]=Σ_{G}P(G)μ_{G}p_{G}=σ_{A}[p(1−p)/2]^{1/2}, and

E[μ_{G}²p_{G}]=Σ_{G}P(G)μ_{G}²p_{G}=(1/2)(1−σ_{R}²)−4p²(1−p)²ad+(2p−1)σ_{D}²/2≈σ_{A}²/2.

The results for Δp,

Δp=2^{1/2}yσ₀σ_{A}/ρσ_{R}, tail pools, and

Δp=[1+X_{T}σ_{A}/2^{3/2}σ₀σ_{R}²]yσ₀σ_{A}/2^{1/2}r(1−r)σ_{R}, affected-unaffected pools,

lead directly to Eqs. 1 and 2.

Approximate genotype relative risks may also be obtained from the Taylor series expansion for θ(G). To lowest order, the relative risk for the heterozygote is approximately 1+(d+a)y/rσ_{R}, and for the A₁A₁ homozygote is 1+2ay/rσ_{R}. For additive inheritance, d=0, and the relative risk is multiplicative with allele dose when ay/rσ_{R} is small. For a complex trait σ_{R} is close to 1, and for a minor allele, a≈σ_{A}/(2p)^{1/2}. When the disease incidence is 10%, the parameter required to be small is 1.24σ_{A}/p^{1/2}.

For individual genotyping, the regression model used to test significance is

X=b₁(p_{G}−p)+ε,

where the residual contribution ε to the phenotype has zero mean and is uncorrelated with p_{G}. Using standard statistical methods (Snedecor, 1989), the test statistic b₁ under the null hypothesis has mean zero and variance Var(b₁|null) given by

Var(b₁|null)=N⁻¹ Var(X)/Var(p_{G})=1/N[p(1−p)/2].

Under the alternative hypothesis, the expectation for the test statistic is

E(b₁)=Cov(X,p_{G})/Var(X)=σ_{A}[p(1−p)/2]^{1/2},

and its variance is

Var(b₁|alt)=N⁻¹ Var(ε)/Var(p_{G})=σ_{R}²/N[p(1−p)/2].

The sample size required for a one-sided test of b₁ with Type I error α and power 1−β is

N=[z_{α}Var(b₁|null)^{1/2}−z_{1-β}Var(b₁|alt)^{1/2}]²/E(b₁)²,

which is the result provided in Eq. 3.

Application of the Methods of the Invention

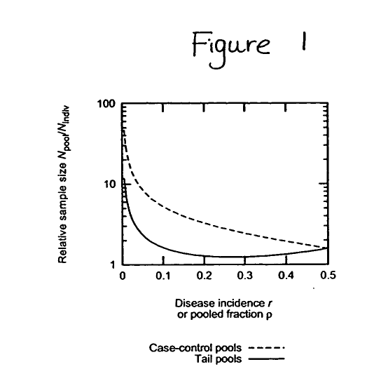

The sample sizes required for the pooled DNA designs are compared in FIG. 1 to the sample size N_{indiv} required by individual genotyping. The ratio N_{c-c}/N_{indiv} (dashed line) is a function of the disease incidence r, while Nₜₐᵢₗ/N_{indiv} (solid line) is a function of the pooling fraction ρ. For typical disease incidence, r˜10%, the affected-unaffected design requires a sample 5.3× larger than that required for individual genotyping. Compared to the tail design, it measures an allele frequency difference that is half as large and is approximately 4× less efficient. The tail design, with ρ=27%, requires a sample only 1.24× larger than required for individual genotyping. The tail design is also robust to variation in ρ near its optimum, as values from 19% to 37% drop the efficiency no more than 5%.

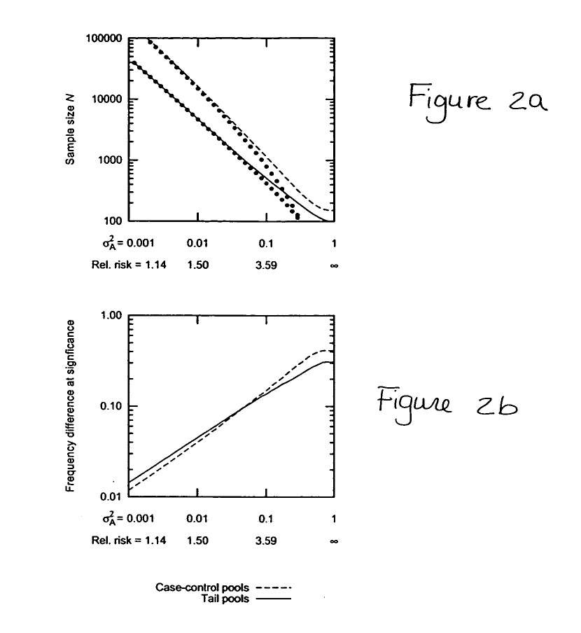

The analytic theory indicates that the additive variance σ_{A}², or equivalently the genotype relative risk for an allele of known frequency, is the most important factor determining the sample size requirements. This dependence is shown in FIG. 2a with exact numerical results for affected-unaffected pools (dashed line) and tail pools (solid line) for type I error of 5×10⁻⁸ and power of 0.8. The minor allele frequency is 10%, its effect on the quantitative phenotype is purely additive, and the disease incidence is 10%. The analytic approximations (solid circles) from Eq. 1 and 2 are nearly indistinguishable from the exact results when the genotype relative risk drops below a factor of 2. As predicted by the analytic theory, the tail pools require smaller sample sizes than the affected-unaffected pools, and the gap grows wider for alleles with a smaller effect on the phenotype. For relative risks of 2 to 5, the deviations from analytic theory are moderate; above a relative risk of 5, the phenotype is monogenic with respect to locus G, and the analytic approximations for complex traits are no longer valid.

The allele frequency difference between pools at the significance threshold is shown in FIG. 2b for affected-unaffected pools (dashed line) and tail pools (solid line). The measurement error in the allele frequency difference must be smaller than the significance threshold to detect association (Darvasi, 1994). Evaluations that provide a frequency difference measurement accurate to 0.04 can detect association with alleles responsible for 1% of the total phenotypic variance, corresponding to a heterozygote relative risk of 1.5. The allele frequency difference measurement must be accurate to 0.01 to detect association with an allele explaining 0.1% of the phenotypic variance, corresponding to a relative risk of 1.14.

To test the range of validity of the analytic estimates for pooling, we performed a series of exact calculations of sample size requirements as a function of p and d/a. Large deviations were seen only when the magnitude of a gene effect μ_{G} approached σ_{R} in size, or, equivalently, when σ_{A}² was larger than the minor allele frequency or when a genotype relative risk was larger than 5 (results not shown). For additive contributions from a minor allele, the range of validity corresponds to σ_{A}²<2p.

The advantages of the methods disclosed herein include the following. The optimal fraction for tail pooling, 27%, is independent of all model parameters including allele frequency, inheritance mode, effect size, and type I error and power, for virtually any QTL contributing to a complex trait. The exceptions to this finding are rare QTLs with relative risks of 5 or greater, and rare, recessive alleles, both of which are more difficult to detect than more frequent alleles contributing to the same overall phenotypic variance. In addition, the tail design is approximately 4-fold more efficient than the affected-unaffected design and requires a sample size only 24% larger than for individual genotyping. Still further, DNA pooling studies designed according to the present procedures disclosed herein provide extremely efficient methods for large-scale screening and should help to make feasible genome-wide association studies.

References

Abecasis, G R, Cardon, L R, Cookson, WOC (2000) A general test of association for quantitative traits in nuclear families. Am J Hum Genet 66: 279-292.

Cargill M, Altshuler D, Ireland J, Sklar P, Ardlie K, Patil N, Shaw N et al. (1999) Characterization of single-nucleotide polymorphisms in coding regions of human genes. Nat Genet 1999 Jul;22(3):231-238.

Collins A, Lonjou C, Morton N E (2000) Genetic epidemiology of single-nucleotide polymorphisms. Proc Natl Acad Sci USA 96:15173-15177.

Daniels, J. K, Holmans, P., Williams, N. M., Turic, D., McGuffin, P., Plomin, R., Owen, M. J. A simple method for analysing microsatellite allele image patterns generated from DNA pools and its application to allelic association studies. Am. J. Hum. Genet. 62, 1189-1197 (1998).

Darvasi A, Soller M (1994) Selective DNA pooling for determination of linkage between a molecular marker and a quantitative trait locus. Genetics 138: 1365-1373.

Falconer, D. S., and MacKay, T. F. C. Introduction to quantitative genetics. (Addison-Wesley, Boston, 1996).

Frank, L (2000) Storm brews over gene bank of Estonian population. Science 286: 1262.

Fulker D W, Cherny S S, Cardon L R (1995) Multipoint interval mapping of quantitative trait loci, using sib pairs. Am J Hum Genet 56:1224-1233.

Fulker, D. W., Cherny, S. S., Sham, P. C., Hewitt, J. K. Combined linkage and association analysis of quantitative traits. Am. J. Hum. Genet. 64, 259-267 (1999).

Hill, W. G. Design and efficiency of selection experiments for estimating genetic parameters. Biometrics 27, 293-311 (1971).

Kimura, M. & Crow, J. F. Effect of overall phenotypic selection on genetic change at individual loci. Proc. Natl. Acad. Sci. USA 75, 6168-6171 (1978).

Kruglyak, L (1999) Prospects for whole-genome linkage disequilibrium mapping of common disease genes. Nature Genetics 22: 139-144.

Liu, B-H (1997) Statistical Genomics. CRC Press, Boca Raton.

Nilsson A, Rose J (1999) Sweden takes steps to protect tissue banks. Science 286: 894.

Ott J (1999) Analysis of human genetic linkage. Johns Hopkins Univ Pr, Baltimore.

Rabinow, P (1999) French DNA: Trouble in Purgatory. University of Chicago Press, Chicago.

Risch, N. J. Searching for genetic determinants in the new millennium. Nature 405, 847-856 (2000).

Risch N J, Merikangas K (1996) The future of genetic studies of complex human diseases. Science 273:1516-1517.

Risch N J, Teng J (1998) The relative power of family-based and case-control designs for linkage disequilibrium studies of complex human diseases I. DNA pooling. Genome Res 8:1273-1288.

Sham, P (1997) Statistics in Human Genetics. Arnold.

Sham, P. C., Chemy, S. S., Purcell, S., Hewitt, J. K. Power of linkage versus association analysis of quantitative traits, by use of variance components models, for sibship data. Am. J. Hum. Genet. 66, 1616-1630 (2000).

Snedecor, G. W., and Cochran, W. G. Statistical Methods, Eighth Edition. (Iowa State University Press, Ames, 1989).

Beyer, W. H. (ed). CRC Standard Mathematical Tables, 27^{th} Edition. (CRC Press, Boca Raton, Fla., 1984).

Press, W. H., Teukolsky, S. A., Vetterling, W. T., and Flannery, B. P. Numerical Recipes in C, The Art of Scientific Computing, Second Edition (Cambridge University Press, Cambridge, UK, 1997).

Chandler, D. Introduction to Modern Statistical Mechanics. (Oxford Univ. Press, New York, 1987).

Ollivier, L., Messer, L. A., Rothschild, M. F. & Legault, C. The use of selection experiments for detecting quantitative trait loci. Genet. Res., Camb. 69, 227-232 (1997).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting an association in a population of unrelated individuals between a genetic locus and a quantitative phenotype, wherein two or more alleles occur at the locus, and wherein the phenotype is expressed using a numerical phenotypic value whose range falls within a first numerical limit and a second numerical limit, the method comprising the steps of
a) obtaining the phenotypic value for each individual in the population;
b) determining the minimum number of individuals from the population required for detecting the association using Eq. 2;
c) selecting a first subpopulation of individuals having phenotypic values that are higher than a predetermined lower limit and pooling DNA from the individuals in the first subpopulation to provide an upper pool;
d) selecting a second subpopulation of individuals having phenotypic values that are lower than a predetermined upper limit and pooling DNA from the individuals in the second subpopulation to provide a lower pool;
e) for one or more genetic loci, measuring the frequency of occurrence of each allele at said locus in the upper pool and the lower pool;
f) for a particular genetic locus, measuring the difference in frequency of occurrence of a specified allele between the upper pool and the lower pool; and
g) determining that an association exists if the allele frequency difference between the pools is larger than a predetermined value.

2. The method of claim 1, wherein the difference in frequency of occurrence of the specified allele has associated with it an error of measurement.

3. The method of claim 2, wherein the error of measurement is 0.04.

4. The method of claim 2, wherein the error of measurement is 0.01.

5. The method described in claim 1, wherein the predetermined lower limit is set so that the upper pool ranges from including the highest 37% of the population to including the highest 19% of the population and the predetermined upper limit is set so that the lower pool ranges from including the lowest 37% of the population to including the lowest 19% of the population.

6. The method of claim 1, wherein the predetermined lower limit is set so that the upper pool includes the highest 27% of the population and the predetermined upper limit is set so that the lower pool includes the lowest 27% of the population.

7. The method of claim 1, wherein the genetic locus has two alleles.

8. The method of claim 1 wherein the population includes individuals who may be classified into classes.

9. The method of claim 8, wherein the classes are based on an age group, gender, race or ethnic origin.

10. The method of claim 8, wherein all the members of a class are included in the pools.

11. The method of claim 1 for determining the genetic basis of disease predisposition.

12. The method of claim 11, wherein the genetic locus which is analyzed for determining the genetic basis of disease predisposition contains a single nucleotide polymorphism.

13. A method for detecting an association in a population of unrelated individuals between a genetic locus and a quantitative phenotype, wherein two or more alleles occur at the locus, and wherein the phenotype is expressed qualitatively as being either affected or unaffected, the method comprising the steps of
a) identifying the phenotype as being either affected or unaffected for each individual in the population;
b) determining the minimum number of individuals from the population required for detecting the association using Eq. 1;
c) pooling all or a portion of the affected individuals into a first pool and all or a portion of the unaffected individuals into a second pool;
d) for one or more genetic loci, measuring the frequency of occurrence of each allele at said locus in the first pool and the second pool;
e) for a particular genetic locus, measuring the difference in frequency of occurrence of a specified allele between the upper pool and the lower pool; and
f) determining that an association exists if the allele frequency difference between the pools is larger than a predetermined value.

14. The method of claim 13, wherein the first pool and second pool have the same number of individuals.

15. The method of claim 13, wherein the difference in frequency of occurrence of the specified allele has associated with it an error of measurement.

16. The method of claim 15, wherein the error of measurement is 0.04.

17. The method of claim 15, wherein the error of measurement is 0.01.

18. The method of claim 13, wherein the genetic locus has two alleles.

19. The method of claim 13, wherein the population includes individuals who may be classified into classes.

20. The method of claim 19, wherein the classes are based on an age group, gender, race or ethnic origin.

21. The method of claim 19, wherein all the members of a class are included in the pools.

22. The method of claim 13 for determining the genetic basis of disease predisposition.

23. The method of claim 22, wherein the genetic locus which is analyzed for determining the genetic basis of disease predisposition contains a single nucleotide polymorphism.
